(19) United States
(12) Patent Application Publication
(10) Pub. No.: US 2003/0224992 A1
(43) Pub. Date: Dec. 4, 2003

(54) Transcription factor modulators and uses thereof

(75) Inventors: John L. Joyal, Melrose, MA (US); Mark A. Findeis, Belmont, MA (US); Malcolm J. Kavarana, Waltham, MA (US); Barry Morgan, Franklin, MA (US); Malcolm L. Gefter, Lincoln, MA (US)

Correspondence Address: LAHIVE & COCKFIELD, 28 STATE STREET, BOSTON, MA, 02109, US

(73) Assignee: Praecis Pharmaceuticals Inc., Waltham, MA

(21) Appl. No.: 10/389,714

(22) Filed: Mar. 14, 2003

Related U.S. Application Data
(60) Provisional application No. 60/365,375, filed on Mar. 15, 2002.
(60) Provisional application No. 60/372,563, filed on Apr. 11, 2002.

(51) Int. Cl. A61K 48/00; A61K 38/17; C07K 14/705; C07K 14/72; C07H 21/04

(52) U.S. Cl. 514/12; 514/44; 530/350; 536/23.5

(57) ABSTRACT

The present invention provides transcription factor modulators useful for modulating gene expression in a cell, as well as pharmaceutical compositions containing these transcription factor modulators. The present invention also provides methods for modulating gene transcription in a cell and methods of treating a subject suffering from a transcription factor-associated disorder, such as cancer, inflammatory disorders or autoimmune disorders.

### RELATED APPLICATIONS

This application claims priority to U.S. Provisional Patent Application Serial No. 60/365,375 filed Mar. 15, 2002 and U.S. Provisional Patent Application Serial No. 60/372,563 filed Apr. 11, 2002, the entire contents of each of which are incorporated herein by reference.

### BACKGROUND OF THE INVENTION

A major focus of cellular and molecular research has concentrated on developing means to regulate gene expression (i.e., gene transcription and translation) in an effort to treat and cure a variety of diseases, such as cancer or inflammatory diseases. The up-regulation or down-regulation of specific genes is believed to alter or circumvent the molecular mechanisms underlying various diseases and conditions.

Currently, several general methods have been developed to regulate and control gene expression at either the transcriptional or translational steps. A significant drawback in each of these methods is the difficulty in delivering the active compound to the nucleus of a cell. Accordingly, there currently exists the need for effective and efficient ways to regulate and control gene expression and, in particular, the need for compounds that are able to reach the nucleus of a cell and modulate gene transcription.

### SUMMARY OF THE INVENTION

The present invention provides transcription factor modulators useful for modulating gene expression in a cell. The present invention is based, at least in part, on the discovery that attaching a transcription control recognition sequence to a membrane permeable peptidic sequence, facilitates the delivery of effective amounts of the transcription control recognition sequence to a nucleus of a cell. The transcription factor modulators of the present invention are particularly suitable for use as pharmaceuticals and, as such, are characterized by specific length limitations (as described herein) and, preferably, by their modified base or sugar moiety content or their modified phosphate backbone content.

Accordingly, in one aspect, the present invention provides a transcription factor modulator which includes a transcription control recognition sequence for a transcription factor coupled, e.g., directly or indirectly, to a membrane permeable peptidic sequence, wherein the membrane permeable peptidic sequence facilitates the delivery of the transcription control recognition sequence to a nucleus of a cell. The transcription control recognition sequence can be linked to the C-terminus or N-terminus of the membrane permeable sequence either directly or via a suitable linker group, and can be double stranded or single stranded.

In one embodiment, the transcription control recognition sequence comprises a promoter element, e.g., a CACCC-Box, a GC-Box, or a CAT-Box. In another embodiment, the transcription control recognition sequence comprises a hormone response element, such as the androgen response element (e.g., AGTACGTGATGTTCT (SEQ ID NO: 10), GAAACAGCCTGTTCT (SEQ ID NO: 11), AGCACTTGCTGTTCT (SEQ ID NO: 12), ATAGCATCTTGTTCT (SEQ ID NO: 13), AGTCCCACTTGTTCT (SEQ ID NO: 14), AGTACTTGTTGTTCT (SEQ ID NO: 15), AGCTCAGCTTGTACT (SEQ ID NO: 16), AGAACAACCTGTTGA (SEQ ID NO: 17), TGAAGTTCCTGTTCT (SEQ ID NO: 18), GTAAAGTACTCCAAGAA (SEQ ID NO: 19), and GGAACAGCAAGTGCT (SEQ ID NO: 20)); the estrogen response element (e.g., GGTCACAGTGACC (SEQ ID NO: 5)); the glucocorticoid response element (e.g., YGGTWCAMWNTGTYCT (SEQ ID NO: 6)); the thyroid hormone response element (e.g., AGGTAAGATCAGGGACGT (SEQ ID NO: 7)); the thyroid hormone inhibitory element (e.g., AGGGTATAAAAAGGGC (SEQ ID NO: 8)); the sterol-dependent repressor (e.g., GTGSGGTG (SEQ ID NO: 9)); or an NF-κB binding site, e.g., GGGRNNTYCC (SEQ ID NO: 21).

The transcription control recognition sequence may include 5-10, 5-20, 5-30, 10-20, 10-30 or 20-30 nucleotides and may, for example, be a double stranded or single stranded oligodeoxynucleotide, a ribonucleic acid, a peptide-nucleic acid, or it may include a modified phosphodiester bond, e.g., phosphorothioate, phosphoramidite, or methyl phosphate derivatives, a modified sugar residue or a modified nucleoside base.

In one embodiment, the membrane permeable peptidic sequence is the Kaposi FGF signal sequence or sequences derived therefrom, the third helix of the antennapedia homeodomain or sequences derived therefrom, the HIV-1 Tat protein or sequences derived therefrom, and the gelsolin sequence or sequences derived therefrom. The membrane permeable peptidic sequence may include 2-10, 2-15, 2-20, 5-20 or 6-20 amino acid residues, e.g., D-amino acid residues, and preferably includes at least one, two, three, four or five basic amino acid residues, e.g., D-amino acid residues.

In another aspect, the present invention provides a pharmaceutical composition which includes a therapeutically effective amount of a transcription factor modulator of the present invention and a pharmaceutically acceptable carrier.

In yet another aspect, the present invention provides a method for modulating gene transcription in a cell. The method includes contacting the cell with a transcription factor modulator of the present invention in a sufficient amount to modulate gene transcription in the cell.

In a further aspect, the present invention provides a method of treating a subject suffering from a transcription factor-associated disorder, such as cancer, for example, a hormonally responsive cancer, e.g., prostate cancer, breast cancer, uterine cancer, or benign prostatic hypertrophy; an NF-κB associated disorder, e.g., inflammatory diseases, autoimmune diseases, ischemic diseases, cachexia, and cancer metastasis and invasion. The method includes administering to the subject a therapeutically effective amount of a transcription factor modulator of the present invention, thereby treating a subject suffering from a transcription factor-associated disorder.

Other features and advantages of the invention will be apparent from the following detailed description and claims.

### BRIEF DESCRIPTION OF THE DRAWINGS

FIG. 1 is a graph depicting the results of the fluorescent polarization assay.

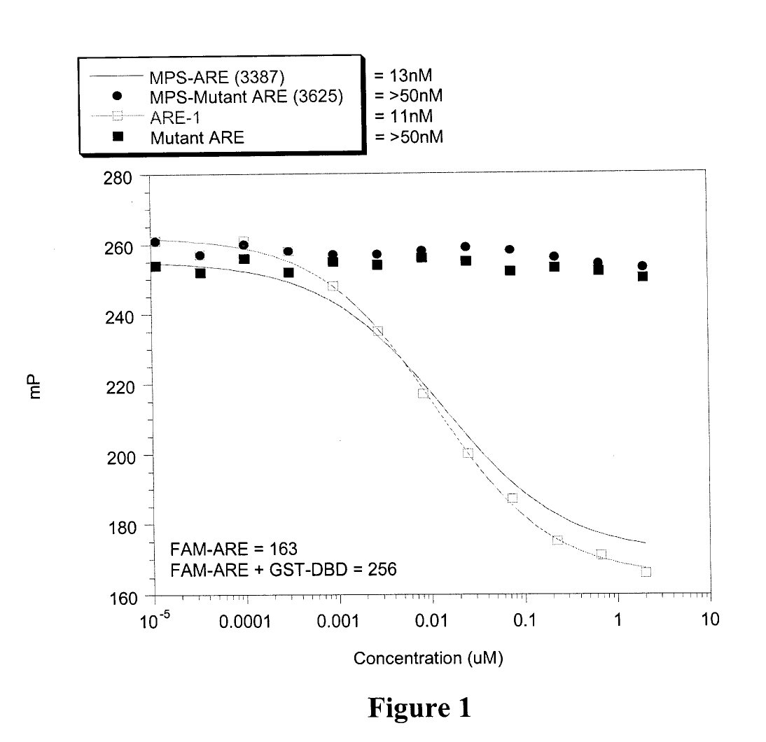

FIG. 2 is a schematic representation of the generation of a transcription factor modulator of the invention containing an androgen response element having the sequence 5′-AGT CTG GTA CAG GGT GTT CTT TTT A-3′ (SEQ ID NO: 23).

FIG. 3 is a reaction scheme for linking an androgen response element oligonucleotide to a membrane permeable peptide sequence.

FIG. 4 is a reaction scheme for linking an androgen response element oligonucleotide to a membrane permeable peptide sequence.

FIG. 5 illustrates the ability of the ARE and ARE-MPS to bind to full length androgen receptor present in LnCAP cell nuclear extracts.

FIG. 6 illustrates a decrease in expression of an androgen receptor responsive gene by LnCAP cells following electroporation of ARE.

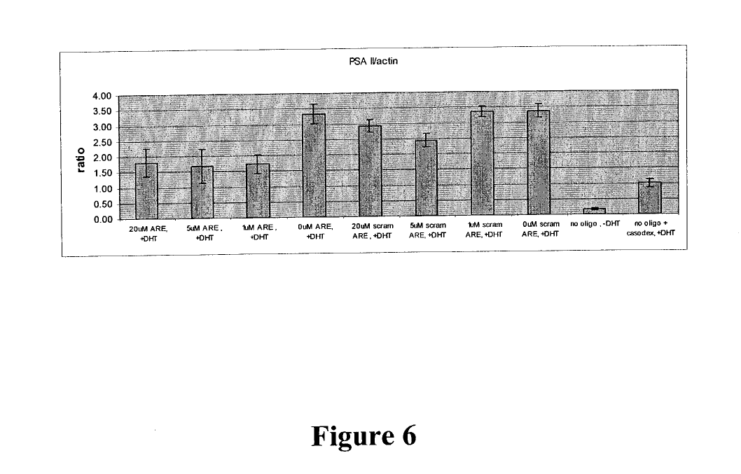

### DETAILED DESCRIPTION OF THE INVENTION

The present invention provides transcription factor modulators useful for modulating gene expression in a cell. The transcription factor modulators of the present invention include a transcription control recognition sequence for a transcription factor, optionally coupled to a membrane permeable peptidic sequence or other chemical moiety capable of enhancing intracellular penetration of the transcription control recognition sequence. Without intending to be bound by theory, it is believed that the membrane permeable peptidic sequence, for example, can facilitate the delivery of the transcription control recognition sequence to a nucleus of a cell.

As used herein, the term “transcription factor modulator” includes the molecules of the invention that bind to or interact with a transcription factor and prevent the binding of the transcription factor to its native transcription control recognition sequence. Transcription factor modulators include a transcription control recognition sequence for a transcription factor coupled to a membrane permeable peptidic sequence, which facilitates the delivery of the transcription control recognition sequence to a nucleus of a cell.

Various aspects of the invention are described further in the following subsections.

I. Transcription Control Recognition Sequences

As used herein, the term “transcription control recognition sequence” or “TCRS” includes a nucleic acid sequence, e.g., a doubled-stranded or single-stranded oligonucleotide or a peptide-nucleic acid, that corresponds to the native DNA binding site for a transcription factor. The TCRS may be identical to or essentially identical to the native sequence, so long as it still retains the ability to bind to the transcription factor. For example, the TCRS may be 90%, 91%, 92%, 93%, 94%, 95%, 96%, 97%, 98% or 99% identical to the native sequence. Transcription control recognition sequences contemplated by the present invention are sequences which are recognized by control proteins, and which are involved in either enhancing or repressing transcription of associated sequences. Transcription control recognition sequences contemplated by the present invention include sequences described in U.S. Pat. No. 5,683,985 and in Locker and Buzard (1990) J. DNA Sequencing and Mapping 1: 3-11, and include promoter elements, hormone response elements, viral and cellular elements, liver associated elements, tissue associated elements, and the like.

Preferred TCRS for use in the transcription factor modulators of the present invention are suitable for use as pharmaceuticals and, thus, are less than 30 nucleotides in length, preferably less than 25 nucleotides in length. For example, the TCRS may be 5, 10, 15, 20, 21, 22, 23, 24, 25, 26, 27, 28 or 29 nucleotides in length.

Exemplary promoter elements that may be used in the transcription factor modulators of the present invention include the CACCC-Box (having the sequence 5′-GCCACACCC-3′ SEQ ID NO: 1), the GC-Box (having the sequence 5′-KRGGCGKRRY-3′, SEQ ID NO: 2, wherein each K is independently G or T; each R is independently G or A; and Y is C or T), the CAT-Box of NF-1 cells (having the sequence 5′-TTGGCNNNNNGCCAA-3′ or 5′-TTGGCNNNNNNGCCA-3′, SEQ ID NO: 3 and 4, respectively, wherein each N is independently selected from A, G, C or T), and the like.

Exemplary hormone response elements include the estrogen response element (having the sequence 5′-GGTCACAGTGACC-3′; SEQ ID NO: 5), the glucocorticoid response element (having the sequence 5′-YGGTWCAMWNTGTYCT-3′, SEQ ID NO: 6, wherein each Y is independently C or T; each W is independently A or T; M is A or C; and N is any one of A, C, G, or T), the thyroid hormone response element (having the sequence 5′-AGGTAAGATCAGGGACGT-3′; SEQ ID NO: 7), the thyroid hormone inhibitory element (having the sequence 5′-AGGGTATAAAAAGGGC-3′; SEQ ID NO: 8), the sterol-dependent repressor (having the sequence 5′-GTGSGGTG-3′ SEQ ID NO: 9, wherein S is G or C), and the like.

Examples of androgen response element that may be used include the following: 5′-AGTACGTGATGTTCT-3′ (SEQ ID NO: 10), 5′-GAAACAGCCTGTTCT-3′ (SEQ ID NO: 11), 5′-AGCACTTGCTGTTCT-3′ (SEQ ID NO: 12), 5′-ATAGCATCTTGTTCT-3′ (SEQ ID NO: 13), 5′-AGTCCCACTTGTTCT-3′ (SEQ ID NO: 14), 5′-AGTACTTGTTGTTCT-3′ (SEQ ID NO: 15), 5′-AGCTCAGCTTGTACT-3′ (SEQ ID NO: 16), 5′-AGAACAACCTGTTGA-3′ (SEQ ID NO: 17), 5′-TGAAGTTCCTGTTCT-3′ (SEQ ID NO: 18), 5′-GTAAAGTACTCCAAGAA-3′ (SEQ ID NO: 19), 5′-GGAACAGCAAGTGCT-3′ (SEQ ID NO: 20) and 5′-AGT CTG GTA CAG GGT GTT CTT TTT A-3′ (SEQ ID NO: 23). Androgen response elements having the consensus sequence 5′-GGAATACANNNTGTTCT-3′ (SEQ ID NO: 22), where each N is any one of A, C, G, or T, may also be used.

NF-κB binding sites may also be used as TCRS. Exemplary NF-κB binding sites that may be used include the sequence 5′-GGGRNNTYCC-3′ (SEQ ID NO: 21) wherein each R is independently G or A; each Y is independently C or T; and each N is any one of A, C, G, or T.

In one embodiment, the TCRS is an RNA molecule. The RNA molecule can be linked to the C-terminus or N-terminus of the membrane permeable sequence either directly or via a suitable linker group, and can be double stranded or single stranded. Preferably, the RNA molecule is a double-stranded RNA molecule and has a sequence which is homologous to at least a portion of a messenger RNA sequence. In this embodiment, the RNA molecule which is linked to the membrane permeable sequence is an interference RNA (‘iRNA”), which can cause destruction of the homologous mRNA and, thereby, prevent or inhibit expression of the protein or peptide encoded by the mRNA.

Typically, a particular mRNA is selected for targeting because it encodes a protein or peptide the expression of which one wishes to prevent or inhibit. For example, the mRNA can encode a protein or peptide which is implicated in a disease or other undesirable medical condition. Examples of proteins and/or peptides which can be encoded by the mRNA include cytokines and lymphokines and their receptors, neurotransmitters and their receptors, oncogenes, growth factors and their receptors, nuclear receptors, transcription factors, and enzymes. Suitable peptides and proteins encoded by the mRNA include, but are not limited to, those set forth in published PCT application WO 01/68836, the entire contents of which are incorporated herein by reference.

The RNA sequence which is linked to the membrane permeable sequence can comprise up to 15, 20, 25, 30, 35, 40, 45, 50, 75, 100, 200, 300 or 400 base pairs. The RNA sequence preferably hybridizes with the target mRNA sequence under conditions of high stringency (e.g., hybridization in 1×SSC, at about 65-70° C. (or hybridization in 1×SSC plus 50% formamide at about 42-50° C.) followed by one or more washes in 0.3×SSC, at about 65-70° C.) and is, preferably, at least about 80%, 85%, 90%, 95%, 98%, 99% or 100% identical to at least a portion of the target mRNA sequence.

As described in further detail below, the transcription control recognition sequence can be modified at the base moiety, sugar moiety or phosphate backbone to improve, e.g., the stability, hybridization, or solubility of the molecule. For example, the deoxyribose phosphate backbone of the transcription control recognition sequence can be modified to generate peptide nucleic acids. The transcription control recognition sequence may also be synthesized with modified phosphodiester bonds, e.g., phosphorothioate, phosphoramidite, and methyl phosphate derivatives.

II. Membrane Permeable Peptidic Sequences

As used herein, the term “membrane permeable peptidic sequence” or “MPS” includes a peptidic sequence which facilitates the transport of a transcription control recognition sequence across cell and/or nuclear membranes. Such peptidic sequences are able to enter cells and/or the nucleus and deliver the transcription control recognition sequence to the cytosol or the nucleus of a cell. Suitable amino acid sequences which may be used in the transcription factor modulators of the present invention are known in the art and include the Kaposi FGF signal sequence (U.S. Pat. No. 5,807,746; U.S. Pat. No. 5,962,415); sequences derived from the HIV TAT protein (U.S. Pat. No. 5,804,604; U.S. Pat. No. 5,670,617 and U.S. Pat. No. 5,747,641), such as the sequence YGRKKRRQRRRP, an active fragment thereof, or a derivative thereof in which from 1 to 12 amino acid residues are D-amino acid residues; the antennapedia homeodomain (U.S. Pat. No. 5,888,762; U.S. Pat. No. 6,080,724) and truncated variants (PCT Application No. WO 00/29427); sequences derived from gelsolin (U.S. Pat. No. 5,846,743; U.S. Pat. No. 5,783,662) and other sequences, as are described in PCT WO 99/29721. Each of the foregoing references is incorporated herein by reference in its entirety.

The membrane permeable peptidic sequence may be at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. Ranges of values using a combination of any of the above recited values as upper and/or lower limits are intended to be included in the present invention.

The membrane permeable peptidic sequence can be linked to the 5′ end or the 3′ end of the transcription control recognition sequence either directly or via a suitable linker group.

As used herein, the terms “peptide compound” and “peptidic sequence/structure” are intended to include peptides comprised of naturally-occurring amino acids, as well as peptide derivatives, peptide analogues and peptide mimetics of the naturally-occurring amino acid structures. The terms “peptide analogue”, “peptide derivative” and “peptidomimetic” as used herein are intended to include molecules which mimic the chemical structure of a peptide and retain the functional properties of the peptide. Approaches to designing peptide analogues, derivatives and mimetics are known in the art. For example, see Farmer, P. S. in Drug Design (E. J. Ariens, ed.) Academic Press, New York, 1980, vol. 10, pp. 119-143; Ball. J. B. and Alewood, P. F. (1990) J. Mol. Recognition 3:55; Morgan, B. A. and Gainor, J. A. (1989) Ann. Rep. Med. Chem. 24:243; and Freidinger, R. M. (1989) Trends Pharmacol. Sci. 10:270.

As used herein, a “derivative” of a compound X (e.g., a peptide or amino acid) refers to a form of X in which one or more reaction groups on the compound have been derivatized with a modifying (derivative) group. Examples of peptide derivatives include peptides in which an amino acid side chain, the peptide backbone, or the amino- or carboxy-terminus has been derivatized (e.g., peptidic compounds with methylated amide linkages).

An “analogue” of a reference amino acid, as the term is used herein, is an α- or β-amino acid having a side chain which is (a) the same as the side chain of the reference amino acid (when the analogue is a β-amino acid residue, a peptoid, or the D-amino acid enantiomer of the reference acid); (b) is an isomer of the side chain of the reference amino acid; (c) is a homologue of the side chain of the reference amino acid; (d) results from replacement of a methylene group in the side chain of the reference amino acid with a heteroatom or group selected from NH, O and S; (e) results from a simple substitution on the side chain of the reference amino acid or any of the preceding (a) to (c); and/or (f) results from a conservative substitution (discussed infra). Analogues of a reference amino acid further include the reference amino acid or any of (a)-(e) above in which the α-nitrogen atom is substituted by a lower alkyl group, preferably a methyl group. A “homologue” of the given amino acid is an α- or β-amino acid having a side chain which differs from the side chain of the given amino acid by the addition or deletion of from 1 to 4 methylene groups. A “simple substitution” of an amino acid side chain results from the substitution of a hydrogen atom in the side chain of the given amino acid with a small substituent, such as a lower alkyl group, preferably a methyl group; a halogen atom, preferably a fluorine, chlorine, bromine or iodine atom; or hydroxy.

Peptide mimetics that are structurally similar to therapeutically useful peptides may be used to produce an equivalent therapeutic or prophylactic effect. The term mimetic, and in particular, peptidomimetic, is intended to include isosteres. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) well known to those skilled in the art. Generally, peptidomimetics are structurally similar to a paradigm peptide (i.e., a peptide that has a biological or pharmacological activity), but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (1980) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982)(—CH(OH)CH2—); Holladay, M. W. et al. (1983) Tetrahedron Lett. 24:4401-4404 (—C(OH)CH2-); and Hruby, V. J. (1982) Life Sci. 31:189-199 (—CH2-S—); each of which is incorporated herein by reference. A particularly preferred non-peptide linkage is —CH2NH—.

Other examples of isosteres include peptides substituted with one or more benzodiazepine molecules (see e.g., James, G. L. et al. (1993) Science 260:1937-1942). Other possible modifications include an N-alkyl (or aryl) substitution (ψ{CONR}), backbone crosslinking to construct lactams and other cyclic structures, substitution of all D-amino acids for all L-amino acids within the compound (“inverso” compounds) or retro-inverso amino acid incorporation (ψ{NHCO}). By “inverso” is meant replacing L-amino acids of a sequence with D-amino acids, and by “retro-inverso” or “enantio-retro” is meant reversing the sequence of the amino acids (“retro”) and replacing the L-amino acids with D-amino acids. For example, if the parent peptide is Thr-Ala-Tyr, the retro modified form is Tyr-Ala-Thr, the inverso form is thr-ala-tyr, and the retro-inverso form is tyr-ala-thr (lower case letters refer to D-amino acids). Compared to the parent peptide, a retro-inverso peptide has a reversed backbone while retaining substantially the original spatial conformation of the side chains, resulting in a retro-inverso isomer with a topology that closely resembles the parent peptide. See Goodman et al. “Perspectives in Peptide Chemistry” pp. 283-294 (1981). See also U.S. Pat. No. 4,522,752 by Sisto for further description of “retro-inverso” peptides. Other derivatives include C-terminal hydroxymethyl derivatives, O-modified derivatives (e.g., C-terminal hydroxymethyl benzyl ether) and N-terminally modified derivatives including substituted amides such as alkylamides and hydrazides.

Such peptide mimetics may have significant advantages over peptide embodiments, including, for example: more economical production, greater chemical stability, enhanced pharmacological properties (e.g., half-life, absorption, potency, efficacy, and the like), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivitization (e.g., labeling) of peptidomimetics should not substantially interfere with the desired biological or pharmacological activity of the peptidomimetic.

Systematic substitution of one or more amino acids of an amino acid sequence with a D-amino acid of the same type (e.g., D-lysine in place of L-lysine) may be used to generate more stable peptides. In addition, constrained peptides may be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The term “conservative substitution”, as used herein, includes the replacement of one amino acid residue by another residue having similar side chain properties. As is known in the art, the twenty naturally amino acids can be grouped according to the physicochemical properties of their side chains. Suitable groupings include alanine, valine, leucine, isoleucine, proline, methionine, phenylalanine and tryptophan (hydrophobic side chains); glycine, serine, threonine, cysteine, tyrosine, asparagine, and glutamine (polar, uncharged side chains); aspartic acid and glutamic acid (acidic side chains) and lysine, arginine and histidine (basic side chains). Another grouping of amino acids is phenylalanine, tryptophan, and tyrosine (aromatic side chains). A conservative substitution involves the substitution of an amino acid with another amino acid from the same group.

Preferred membrane permeable peptidic sequences for use in the transcription factor modulators of the present invention are suitable for use as pharmaceuticals and, thus, preferably include at least one D-amino acid residue (to generate a more stable peptide). In another embodiment, the membrane permeable sequence consists solely of D-amino acid residues.

III. Additional Transcription Factor Modulators

In certain embodiments, the transcription factor modulators of the invention do not include a membrane permeable sequence. In these embodiments, the transcription factor modulators comprise a transcription control recognition sequence, such as an androgen response element as described above, and, optionally, can further include one or more modifying moieties other than an MPS conjugated, directly or via a suitable linker, to the TCRS. Such modifying moieties are selected so as to enhance the biological activity of the transcription factor modulator. For example, the modifying moiety or moieties can be selected to enhance the bioavailability, biodistribution and/or cellular uptake of the compound. Suitable modifying moieties are described, for example, in published PCT application WO 93/07883, the contents of which are incorporated herein by reference in their entirety. Such modifying moieties include, but are not limited to, lipid moieties, such as a cholesterol moiety (Letsinger et al., Proc. Natl. Acad. Sci. USA, 1989, 86, 6553-6556), a cholic acid or cholyl moiety (Manoharan et al., Bioorg. Med. Chem. Let., 1994, 4, 1053-1060), a thioether moiety (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, such as an dodecandiol, decyl, dodecyl or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or an adamantane acetic acid moiety (Manoharan et al., Tetrahedron Lett., 1995, 36, 3651-3654), a fatty acid or fatty acyl moiety, such as a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). The contents of each of the foregoing references are hereby incorporated by reference in their entirety. The TCRS can be modified with the modifying moiety or moieties at the 5′-end, the 3′-end or elsewhere.

In a preferred embodiment, the transcription factor modulator of the invention comprises an androgen response element, as described above. In this embodiment, the androgen response element is, optionally, directly or indirectly conjugated to one or more modifying moieties.

IV. Preparation of the Transcription Factor Modulators

The transcription factor modulators of the present invention are prepared by linking the membrane permeable peptidic sequence to the transcription control recognition sequence. The membrane permeable peptidic sequence may be linked to either the 5′ end or the 3′ end of the transcription control recognition sequence directly or indirectly, e.g., via a suitable linker group (as described below). Similarly, the transcription control recognition sequence may be linked to either the N-terminus or the C-terminus of the membrane permeable peptidic sequence directly or indirectly, e.g., via a suitable linker group (as described below).

The membrane permeable peptidic sequence may be prepared by any of the any well known methods for preparing peptidic sequences. For example, the membrane permeable peptidic sequences may be produced in prokaryotic or eukaryotic host cells by expression of polynucleotides encoding the particular peptide sequence. Alternatively, such membrane permeable peptidic sequences may be synthesized by chemical methods. Methods for expression of heterologous peptides in recombinant hosts, chemical synthesis of peptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken, I. M. (1981) CRC Crit. Rev. Biochem. 11:255; Kaiser et al. (1989) Science 243:187; Merrifield, B. (1986) Science 232:342; Kent, S. B. H. (1988) Ann. Rev. Biochem. 57:957; and Offord, R. E. (1980) Semisynthetic Proteins, Wiley Publishing, which are incorporated herein in their entirety by reference).

The membrane permeable peptidic sequences may also be prepared by any suitable method for chemical peptide synthesis, including solution-phase and solid-phase chemical synthesis. Preferably, the peptides are synthesized on a solid support. Methods for chemically synthesizing peptides are well known in the art (see, e.g., Bodansky, M. Principles of Peptide Synthesis, Springer Verlag, Berlin (1993) and Grant, G. A (ed.). Synthetic Peptides: A User's Guide, W. H. Freeman and Company, New York (1992). Automated peptide synthesizers useful to make the membrane permeable peptidic sequences are commercially available.

The membrane permeable peptidic sequences can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, may be used when synthesizing the membrane permeable peptidic sequences. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others.

The transcription control recognition sequence may be prepared by standard synthetic techniques, e.g., using an automated DNA synthesizer (such as, for Example, Applied Biosystems Model 394). Alternatively, PCR amplification of gene fragments can be carried out to generate a sequence of interest (see, for example, Current Protocols in Molecular Biology, eds. Ausubel et al. John Wiley & Sons: 1992). The transcription control recognition sequence may be synthesized as a double-stranded or a single-stranded molecule. In one embodiment, the transcription control recognition sequence is synthesized as a single-stranded molecule, it is subsequently coupled to the membrane permeable peptidic sequence and then annealed with a complementary sequence to form a transcription factor modulator. In certain embodiments, the transcription control recognition sequence within the transcription factor modulator is single stranded.

In another embodiment, the transcription control recognition sequence is a double-stranded molecule, but the two strands are covalently linked. For example, the two strands can result from self-annealing of a single strand comprising both a TCRS nucleiotide sequence and the reverse complement of the TCRS sequence. The two sequences are, preferably, separated by a third nucleotide sequence sufficient to form a hairpin formation allowing the TCRS sequence and its reverse complement to anneal to form a monomolecular double-stranded oligonucleotide. The TCRS sequence and its reverse complement can also be coupled together using a suitable linking group having sufficient flexibility to allow monomolecular annealing of the TCRS sequence and its complement.

In another embodiment, the transcription control recognition sequence can be modified at the base moiety, sugar moiety or phosphate backbone to improve, e.g., the stability, hybridization, or solubility of the molecule. For example, the deoxyribose phosphate backbone of the transcription control recognition sequence can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. (1996) Proc. Natl. Acad. Sci. 93:14670-675 and U.S. Pat. No. 6,228,982, the contents of each of which are incorporated herein by reference.

In another embodiment, the transcription control recognition sequence can be synthesized with modified phosphodiester bonds, modified nucleobases and/or modified sugar residue, as is known in the art. Suitable modifications are described, for example, in U.S. Pat. No. 6,329,203, the teachings of which are incorporated herein by reference. For example, the phosphodiester backbone of the oligonucleotide can be replaced with moieties including, but not limited to, phosphorothioates, chiral phosphorothioates, phosphorodithioates, phosphorothioamidates, phosphotriesters, aminoalkylphosphotriesters, methyl and other alkyl phosphonates including 3′-alkylene phosphonates, 5′-alkylene phosphonates and chiral phosphonates, phosphinates, phosphoramidates, such as 3′-amino phosphoramidate and aminoalkylphosphoramidates, thionophosphoramidates, thionoalkylphosphonates, thionoalkylphosphotriesters, selenophosphates and boranophosphates. The oligonucleotides can also have backbones formed by groups such as short chain alkylene or cycloalkylene groups, heteroalkyl or heterocycloalkyl internucleoside linkages; siloxane groups; sulfide, sulfoxide, sulfonate, sulfonamide and sulfone groups; formacetyl and thioformacetyl groups; methylene formacetyl and thioformacetyl groups; riboacetyl groups; alkene containing groups; sulfamate groups; methyleneimino and methylenehydrazino groups; and amide groups.

Other modified oligonucleotides contain one or more substituted sugar moieties, such as sugar moieties including substituents such as hydroxy; fluoro; O-alkyl, S-alkyl, NH-alkyl; O-alkenyl, S-alkenyl, NH-alkenyl; O-alkynyl, S-alkynyl or NH-alkynyl; O-alkyl-O-alkyl, C1-C6 alkyl, substituted C1-C6 alkyl, alkenyl, alkynyl, alkaryl, aralkyl, O-alkylaryl, O-arylalkyl, SH, S-alkyl, OCN, Cl, Br, CN, CF₃, OCF₃, SO-alkyl, SO₂-alkyl, ONO₂, NO₂, azido, amino, substituted amino, cycloalkyl, cycloalkylaryl, heterocycloalkyl, heterocycloalkylaryl, and aminoalkylamino.

Suitable sugar modifications further include Locked Nucleic Acids (LNAs), in which 2′-hydroxyl group is covalently linked to either the 3′ or 4′ carbon atom of the sugar ring thereby forming a bicyclic moiety. Preferably, the 2′ oxygen atom and the 4′ carbon atom are linked via a methylene group. Such Locked Nucleic Acids are described in published PCT applications WO 98/39352 and WO 99/14226, the teachings of both of which are hereby incorporated by reference in their entirety.

The oligonucleotides of the invention can also include modified or substituted bases. For example, the oligonucleotides can include substituted derivatives of adenine, guanine, thymine, cytosine and uracil, and aza- and deaza-derivatives of these bases. Suitable derivatives include 5-methylcytosine; 5-hydroxymethylcytosine; xanthine; hypoxanthine; 2-aminoadenine; alkyl-substituted adenine, guanine, thymine, cytosine and uracil; alkynyl-substituted pyrimidine bases, such a 5-propynyluracil and 5-propynylcytosine; 6-azouracil, 6-azocytosine; 6-azothymine, 5-uracil (pseudouracil), 4-thiouracil; 8-halo-, 8-amino-, 8-mercapto-, 8-thioalkyl- and 8-hydroxyladenine and -guanine; 5-substituted uracils and cytosines, such as 5-halouracil and -cytosine; 7-methylguanine, 7-methyladenine, 2-fluoroadenine, and 2-aminoadenine. Suitable aza- and deaza derivatives include 8-azaguanine, 8-azaadenine, 7-deazaguanine, 7-deazaadenine, 3-deazaguanine and 3-deazaadenine.

The transcription control recognition sequence may also be a ribonucleic acid, e.g., an interference RNA (‘iRNA”), which can cause destruction of the homologous mRNA and thereby prevent or inhibit expression of the protein or peptide encoded by the mRNA (as described above).

The membrane permeable peptidic sequence may be linked to the transcription control recognition sequence by any means which produces a stable link between the two and which does not alter the function of each constituent. Preferably, the link between the membrane permeable peptidic sequence and the transcription control recognition sequence is covalent. The membrane permeable peptidic sequence may be linked to either the 5′ end or the 3′ end of the transcription control recognition sequence directly or indirectly. Similarly, the transcription control recognition sequence may be linked to either the N-terminus or the C-terminus of the membrane permeable peptidic sequence directly or indirectly.

Coupling of the membrane permeable peptidic sequence and the TCRS can be accomplished via a coupling or conjugating agent. There are several intermolecular cross-linking reagents which can be used (see, for example, Means, G. E. and Feeney, R. E., Chemical Modification of Proteins, Holden-Day, 1974, pp. 39-43). Among these reagents are, for example, J-succinimidyl 3-(2-pyridyldithio) propionate (SPDP) or N, N′-(1,3-phenylene) bismaleimide (both of which are highly specific for sulhydryl groups and form irreversible linkages); N, N′-ethylene-bis-(iodoacetamide) or other such reagent having 6 to 11 carbon methylene bridges (which relatively specific for sulfhydryl groups); and 1,5-difluoro-2,4-dinitrobenzene (which forms irreversible linkages with amino and tyrosine groups). Other cross-linking reagents useful for this purpose include: p,p′-difluoro-m, m′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine).

Cross-linking reagents may be homobifunctional, i.e., having two functional groups that undergo the same reaction. A preferred homobifinctional cross-linking reagent is bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain.

Cross-linking reagents may also be heterobifunctional. Heterobifunctional cross-linking agents have two different functional groups, for example an amine-reactive group and a thiol-reactive group, that will cross-link two proteins having free amines and thiols, respectively. Examples of heterobifunctional cross-linking agents are succinimidyl 4-(N-maleimidomethyl)cyclohexane-1-carboxylate (“SMCC”), m-maleimidobenzoyl-N-hydroxysuccinimide ester (“MBS”), and succinimide 4-(p-maleimidophenyl)butyrate (“SMPB”), an extended chain analog of MBS. The succinimidyl group of these cross-linkers reacts with a primary amine, and the thiol-reactive maleimide forms a covalent bond with the thiol of a cysteine residue.

Cross-linking reagents often have low solubility in water. A hydrophilic moiety, such as a sulfonate group, may be added to the cross-linking reagent to improve its water solubility. Sulfo-MBS and sulfo-SMCC are examples of cross-linking reagents modified for water solubility.

Many cross-linking reagents yield a conjugate that is essentially non-clearable under cellular conditions. However, some cross-linking reagents contain a covalent bond, such as a disulfide, that is clearable under cellular conditions. For example, dithiobis(succinimidylpropionate) (“DSP”), Traut's reagent and N-succinimidyl 3-(2-pyridyldithio)propionate (“SPDP”) are well-known cleavable cross-linkers. The use of a clearable cross-linking reagent permits the TCRS to separate from the membrane permeable peptidic sequence after delivery into the target cell. Direct disulfide linkage may also be useful.

Some new cross-linking reagents such as n-γ-maleimidobutyryloxy-succinimide ester (“GMBS”) and sulfo-GMBS, have reduced immunogenicity. In some embodiments of the present invention, such reduced immunogenicity may be advantageous.

Numerous cross-linking reagents, including the ones discussed above, are commercially available and described in, for example, S. S. Wong, Chemistry of Protein Conjugation and Cross-Linking, CRC Press (1991).

Chemical cross-linking may include the use of spacer arms. Spacer arms provide intramolecular flexibility or adjust intramolecular distances between conjugated moieties and thereby may help preserve biological activity. A spacer arm may be in the form of a polypeptide moiety comprising spacer amino acids. Alternatively, a spacer arm may be part of the cross-linking reagent, such as in “long-chain SPDP” (Pierce Chem. Co., Rockford, Ill., cat. No. 21651 H).

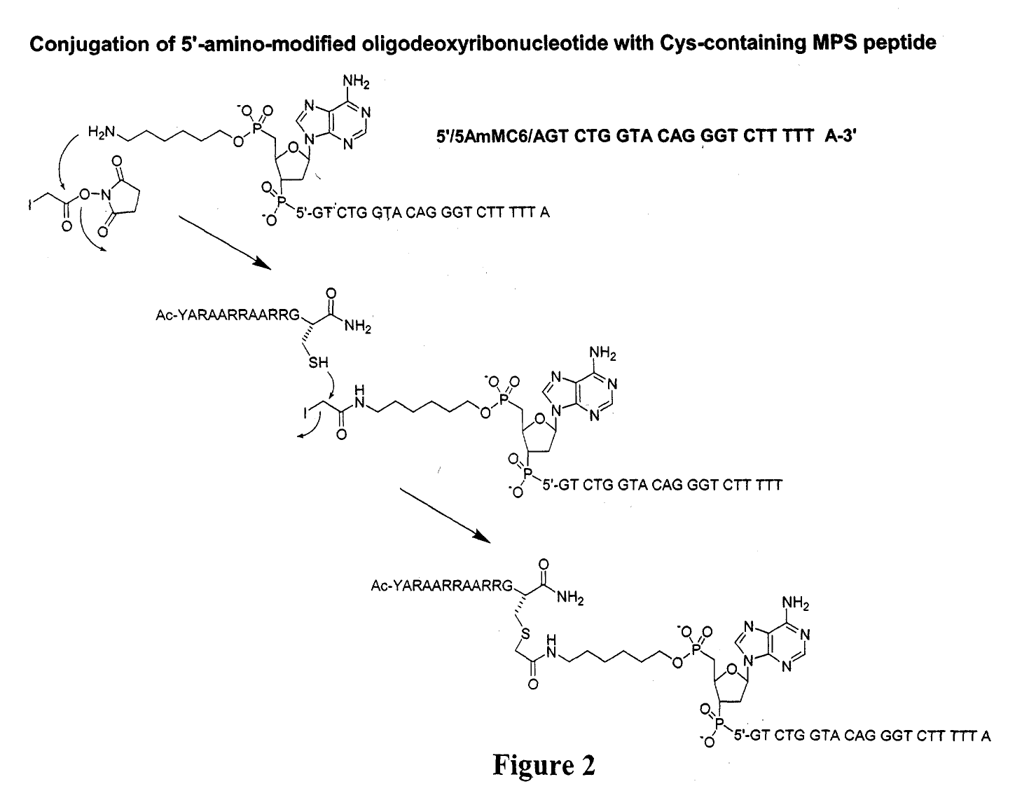

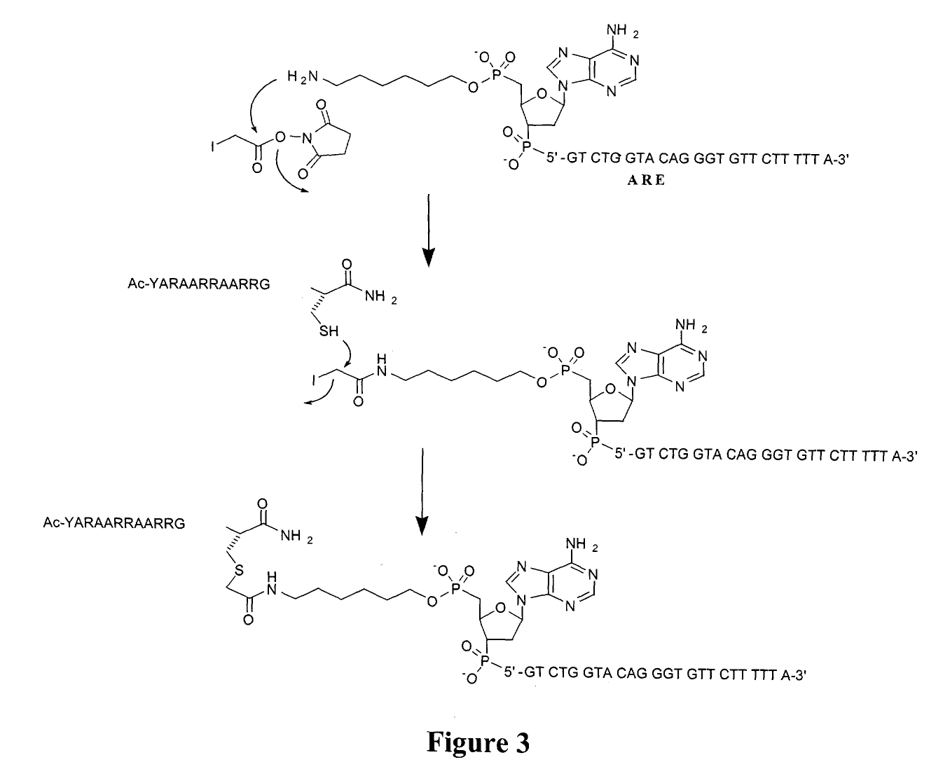

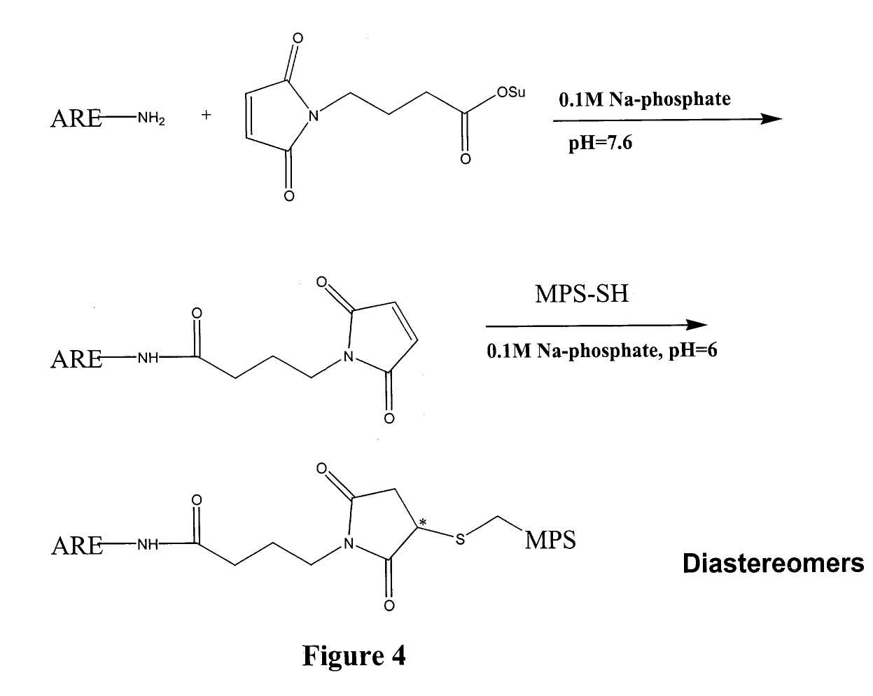

Examples of suitable methods for linking the MPS and the TCRS are provided in FIGS. 2, 3 and 4 and Example 1. FIGS. 2 and 3 show general methods for linking an MPS comprising a free thiol group to an oligonucleotide which has been derivatized at the 5′ or 3′ end by addition of a 4-maleimidobutanoyl group. The MPS can be any MPS comprising a free thiol group, such as a cysteine-containing MPS, or an MPS to which a cysteine or D-cysteine residue has been added, optionally in combination with one or more additional residues, or an MPS which has been derivatized, for example, at the C- or N-terminus, with a moiety other than cysteine which comprises a free thiol group.

V. Pharmaceutical Compositions Containing Transcription Factor Modulators

Another aspect of the invention pertains to pharmaceutical compositions of the transcription factor modulators of the invention. The pharmaceutical compositions of the invention typically comprise a transcription factor modulator of the invention and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, monostearate salts and gelatin. Moreover, the compounds can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are generally known to those skilled in the art.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Depending on the route of administration, the compound may be coated in a material to protect it from the action of enzymes, acids and other natural conditions which may inactivate the agent. For example, the compound can be administered to a subject in an appropriate carrier or diluent co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluoro-phosphate (DEP) and trasylol. Liposomes include water-in-oil-in-water emulsions as well as conventional liposomes (Strejan, et al., (1984) J. Neuroimmunol 7:27). Dispersions can also be prepared in glycerol, liquid polyethylene glycols, and mixtures thereof and in oils. Under ordinary conditions of storage and use, these preparations may contain a preservative to prevent the growth of microorganisms.

The active agent in the composition (i.e., a transcription factor modulator of the invention) preferably is formulated in the composition in a therapeutically effective amount. A “therapeutically effective amount” refers to an amount effective, at dosages and for periods of time necessary, to achieve the desired therapeutic result, such as modulation of androgen receptor activity to thereby influence the therapeutic course of a particular disease state. A therapeutically effective amount of an active agent may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. In another embodiment, the active agent is formulated in the composition in a prophylactically effective amount. A “prophylactically effective amount” refers to an amount effective, at dosages and for periods of time necessary, to achieve the desired prophylactic result, for example, modulation of androgen receptor activity for prophylactic purposes. Typically, since a prophylactic dose is used in subjects prior to or at an earlier stage of disease, the prophylactically effective amount will be less than the therapeutically effective amount.

The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

A transcription factor modulator of the invention can be formulated into a pharmaceutical composition wherein the compound is the only active agent therein. Alternatively, the pharmaceutical composition can contain additional active agents. For example, two or more peptide compounds of the invention may be used in combination.

VI. Methods for Using the Transcription Factor Modulators

The present invention also provides a method for modulating gene transcription in a cell, e.g., a cell within a subject. The method includes contacting a cell with a transcription factor modulator of the invention in an amount sufficient to modulate gene transcription in a cell.

The present invention also provides a method of treating a subject suffering from a transcription factor-associated disorder. The method includes administering to the subject a therapeutically effective amount of a transcription factor modulator of the invention, thereby treating a subject suffering from a transcription factor-associated disorder.

As used herein, a “transcription factor-associated disorder” includes a disease, disorder, or condition, which is caused or associated with the function of a transcription factor in a cell. A transcription factor-associated disorder includes a disease, disorder, or condition, which proceeds, directly or indirectly, via transcription factor-induced gene transcription.

For example, a transcription factor-associated disorder may be an NF-κB associated disorder, such as: (a) an ischemic disease, e.g., ischemic diseases of organs (e.g., ischemic heart diseases such as myocardial infarction, acute heart failure, chronic heart failure, ischemic brain diseases such as cerebral infarction, and ischemic lung diseases such as pulmonary infarction), aggravation of the prognosis of organ transplantation or organ surgery (e.g., aggravation of the prognosis of heart transplantation, cardiac surgery, kidney transplantation, renal surgery, liver transplantation, hepatic surgery, bone marrow transplantation, skin grafting, corneal transplantation, and lung transplantation), reperfusion disorders, and post-PTCA restenosis; (b) an inflammatory disease, e.g., nephritis, hepatitis, arthritis, acute renal failure, chronic renal failure, and arteriosclerosis; and (c) an autoimmune disease, e.g., rheumatism, multiple sclerosis, and Hashimoto's thyroiditis. An NF-κB containing transcription factor modulator of the present invention is particularly suited for the therapy and prophylaxis of reperfusion disorders in ischemic diseases, aggravation of the prognosis of organ transplantation or organ surgery, post-PTCA restenosis, cancer metastasis and invasion, and cachexia such as weight loss following the onset of a cancer.

A transcription factor-associated disorder may also be an androgen-associated disorder, i.e., a disease, disorder, or condition, which proceeds, directly or indirectly, via androgen receptor-induced gene transcription. Androgen associated disorders include benign prostatic hypertrophy, male pattern baldness, acne, idiopathic hirsutism, and Stein-Leventhal syndrome. Androgen associated disorders further include cancers whose growth is promoted by Androgens. Examples of Androgen promoted cancers include prostate cancer (Mendelson (2000) Prog Drug Res 55:213-33), ovarian cancer (Ilekis, et al. (1997) Gynecol Oncol. 66(2):250-4), bladder cancer (Zhuang, et al. (1997) Histopathology 30(6):556-62), colon cancer (Catalano, et al. (2000) Int. J. Cancer 86(3):325-30), liver cancer (Cui (1995) Chung Hua Chung Liu Tsa Chih 17(4):304-6), endometrial cancer (Hackenberg, et al. (1994) Int. J. Cancer 57(1):117-22), pancreatic cancer (Greenway (2000) Drugs Aging 17(3):161-3), lung cancer (Maasberg, et al. (1989) Int. J. Cancer 43(4):685-91), esophageal cancer (Yamashita, et al. (1989) Jpn J Surg 19(2):195-202), cancer of the larynx (Marugo, et al. (1987) J Endocrinol Invest 10(5):465-70), and breast cancer. Other androgen-associated disorders include androgen insensitivity syndromes, infertility, endometrial cancer, and X-linked spinal bulbar muscular atrophy (SMBA). Examples of partial androgen insensitivity syndromes include incomplete testicular feminization, Reifenstein syndrome, Lubs syndrome, Gilbert-Dreifus syndrome, and Rosewater syndrome.

A transcription factor-associated disorder may also be an estrogen receptor-associated disorder, i.e., a disease, disorder, or condition, which proceeds, directly or indirectly, via estrogen receptor-induced gene transcription. Examples of estrogen receptor-associated disorders include breast cancer, osteoporosis, endometriosis, cardiovascular disease, hypercholesterolemia, prostatic hypertrophy, prostatic carcinomas, obesity, hot flashes, skin effects, mood swings, memory loss, menopausal syndromes, hair loss (alopecia), type-II diabetes, Alzheimer's disease, urinary incontinence, GI tract conditions, spermatogenesis, disorders associated with plasma lipid levels, acne, hirsutism, other solid cancers (such as colon, lung, ovarian, testis, melanoma, CNS, and renal), multiple myeloma, cataracts, lymphoma, and adverse reproductive effects associated with exposure to environmental chemicals.

As used herein, the term “subject” includes warm-blooded animals, preferably mammals, including humans. In a preferred embodiment, the subject is a primate. In an even more preferred embodiment, the primate is a human.

As used herein, the term “administering” to a subject includes dispensing, delivering or applying a transcription factor modulator of the invention e.g., a transcription factor modulator in a pharmaceutical formulation, to a subject by any suitable route for delivery of the composition to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route.

As used herein, the term “therapeutically effective amount” includes an amount effective, at dosages and for periods of time necessary, to achieve the desired result, e.g., sufficient to treat a transcription factor-associated disorder in a subject. An effective mamount of a transcription factor modulator of the invention, as defined herein may vary according to factors such as the disease state, age, and weight of the subject, and the ability of the peptide compound to elicit a desired response in the subject. Dosage regimens may be adjusted to provide the optimum therapeutic response. An effective amount is also one in which any toxic or detrimental effects (e.g., side effects) of the peptide compound are outweighed by the therapeutically beneficial effects.

VII. Screening Assays

The invention also provides a method of assessing the ability of a transcription factor modulator to bind to a transcription factor within a cell and, thereby, prevent the binding of the transcription factor to its native transcription control recognition sequence. The method includes (1) providing a cell which contains the native transcription control recognition sequence for the transcription factor attached to a reporter gene; (2) contacting the cell with a transcription factor modulator of the present invention; (3) and monitoring expression of the reporter gene to, thereby, assess the ability of the transcription factor modulator to bind to a transcription factor within a cell and prevent the binding of the transcription factor to its native transcription control recognition sequence (and drive the expression of the reporter gene).

The reporter gene that is used may be any commercially available reporter gene, e.g., chloramphenicol acetyltransferase (CAT) or luciferase. A luciferase reporter assay, e.g., the Dual-Luciferase™ Reporter Assay (available through Promega and described in Sherf, B. A. et al. (1996) Promega Notes Magazine 57:02); and the secreted alkaline phosphatase (SEAP) assay described in, for example, Berger, J. et al. Gene 66:1-10, the contents of which are incorporated herein by reference, may be used.

To determine whether a transcription factor modulator of the present invention binds to a transcription factor within a cell, cells may be contacted with the transcription factor modulator and nuclear extracts prepared using art known techniques (e.g., using the method of Dignam et al. (1983) Nucleic Acid Res. 11, 1475, the contents of which are incorporated herein by reference). The nuclear extracts may then be analyzed using a mobility shift assay (e.g., the one described by Fried and Crothers (1981) Nucleic Acid Res. 9, 6506) to, for example, determine the percentage inhibition of transcription factor binding to a radiolabeled transcription control recognition sequence.

The invention is further illustrated by the following examples, which should not be construed as further limiting. The contents of all references, pending patent applications and published patents cited throughout this application, as well as the Figures and the Sequence Listing, are hereby expressly incorporated by reference.

### EXAMPLES

In each of the following examples, the term “ARE” refers to the sequence of SEQ ID NO: 23. The scrambled ARE sequence, referred to as AREsc is 5′-AGT CTA CGA GTG GGG TTC TTT TTT A-3′ (SEQ ID NO: 24).

### Example 1

Synthesis of Transcription Factor Modulators

The Transcription Factor Modulators of the present invention may be generated using the following procedure, which was used to generate the Transcription Factor Modulator, as shown schematically in FIG. 2.

An aqueous solution of the oligonucleotide (4.13 mM, 82.6 nmoles) was basified by the addition of saturated ammonium bicarbonate to a pH=9.0 by damp pH paper. To this solution was added N-Succimidyl Iodoacetate (SIA) in dimethylformamide (166 nmoles), and the pH rechecked by damp pH paper. After 40 minutes at room temperature, the progress of the reaction was checked by reverse-phase high performance liquid chromatography (Hamilton PRP-1 RP-column). A gradient of triethylammonium acetate (pH=7.15, 0.06M) and acetonitrile (5% CH₃CN—30% CH₃CN over 25 min) was used as the eluent; Retention times: unreacted oligonucleotide 11.4 min. and oligonucleotide-SIA adduct 12.7 min. When greater than 70% of the oligonucleotide has reacted with SIA (based on intergrated intensities of the HPLC peaks), an aqueous solution of the MPS-peptide (Ac-YARAARRAARRGC-NH₂; 166 nmoles) was added to the reaction mixture, which was then allowed to stand at room temperature for 4-5 hours. It should be noted that the pH of this solution was checked periodically over the course of the reaction by damp ninhydrin paper and was maintained at a pH=9.0 by the addition of an aqueous solution of saturated sodium bicarbonate. The progress of the coupling reaction was determined from the integrated intensity of the product peak (retention time: 13.96 min) using RP-HPLC. After completion of the coupling reaction, the reaction mixture was acidified by the addition of 1% aqueous trifluoroacetic acid (TFA) to a pH of 6.0 and then lyophilized. The crude product was purified to >99% homogeneity by RP-HPLC and the fractions corresponding to the product peak pooled and lyophilized to dryness. The identity of the desired product was confirmed by matrix assisted laser desorption-ionization mass spectrometry (MALDI) using a Applied Biosystems Voyager System 6097 spectrometer. The sample was premixed with a matrix consisting of 3-hydroxypicolinic acid (50 mg/ml) and diammonium citrate (50 mg/ml; 9:1) and dried under a stream of cold air; Expected MH⁺=9446.9, Found: 9447.0.

A similar reaction performed using the MPS Ac-YGRKKRRQRRRPC-NH₂ also yielded the expected MPS-ARE conjugate, as determined by MALDI mass spectrometry.

### Example 2

Fluorescence Polarization Assay

To test the ability of the Transcription Factor Modulators of the present invention to bind to their target DNA binding site, an androgen responsive element having the sequence 5′-AGT CTG GTA CAG GGT GTT CTT TTT A-3′ (SEQ ID NO: 23) was attached to a commercially available fluorescent label, FAM. The ability of the transcription factor modulator of Example 1 (ARE-MPS, MPS=Ac-YARAARRAARRGC-NH₂) and the ARE alone to bind the androgen receptor DNA binding site was compared to that of control compounds having a scrambled ARE nucleotide sequence, either alone (AREsc) or linked to the same MPS (AREsc-MPS).

FAM-ARE oligos were annealed by mixing 1 μL of FAM-ARE (5′-/56-FAM/AGT CTG GTA CAG GGT GTT CTT TTT A-3′) and 1.2 μL bottom (5′-TAA AAA GAA CAC CCT GTA CCA GAC T-3′) with 82.8 μL of dH20, 10 μL 1N NaCl, and 5 μL 1M Hepes in a PCR tube. The sample was heated in a PCR machine to 95° C. for 15 minutes. The sample was then transferred to an eppendorf tube and incubated at room temperature for 30-60 minutes. This stock solution had a concentration of 10 uM. During the foregoing incubation, the binding buffer required for the assay (which includes 15 mM Hepes, pH 7.9, 50 mM KCl, 2.5 mM MgCl2, 5% glycerol, 0.1% Triton-X, 1 mM DTT, 1 μg/ml poly dI/dC, 0.1 mg/ml Ac-BSA) was prepared. The modulators to be tested were also prepared as 10 mM DMSO stocks.

For FAM-ARE/GST-DBD/Peptide Wells

1 tube of 1 nM FAM-ARE was prepared in 37 ml of binding buffer and 50 nM of GST-ARDBD (androgen receptor binding domain) were added. In a round bottom polypropylene 96 well plate, 300 μl of FAM-ARE/GST-DBD mixtures were added to column 1 and 150 μl of FAM-ARE/GST-DBD (DNA binding domain) mixtures were added to columns 2-12. 1.5 μl of the ARE, ARE-MPS, AREsc or AREsc-MPS stock solution was then added into column one. 150 μl were transferred across the plate and the last 150 μl were discarded. 40 μl/well (in triplicate) were transferred into a 384 black well plate. Samples were then added to A1-12, B1-12, C1-12 in triplicate. The next sample was then added in wells E1-G12, I1-K12, and M1-O12. The Equalizer 384 from Matrix was used to transfer the solutions in the 96 well plate to the 384 well plate.

For Control One: FAM-ARE (Without GST-ARDBD) Plus Peptide Wells

One tube of 1 nM FAM-ARE was prepared in 37 ml of binding buffer. In a separate 96 well round bottom polypropylene plate 200 μl of FAM-ARE were added to column 1 and 100 μl were added to columns 2-12. 1 μl of ARE-MPS, ARE, AREsc or AREsc-MPS was added to column one and 100 μl were transferred across the plate and the last 100 μl were discarded. 40 μl of sample per well were transferred into a 384 black well plate into A13-A24, E13-24, I13-24, and M13.24.

For Control Two: Binding Buffer with Modulator Compound (no FAM-ARE or GST-ARDBD)

In a third 96 well plate, 200 μl of binding buffer was added to column 1 and 100 μl were added to columns 2-12. Subsequently, 1 μl of ARE-MPS, ARE, AREsc or AREsc-MPS stock solution was added to column one and 100 μl were transferred across the plate and the last 100 μl were discarded. 40 μl of sample per well were transferred into a 384 black well plate into D1-12, H1-12, L1-12, P1-12.

For Control Three: FAM-ARE Only

40 μl of FAM-ARE (per well) were transferred into the 384 black well plate into wells B13, B14, B15.

For Control Four: FAM-ARE+GST-ARDBD Only

40 μl of FAM-ARE+GST-ARDBD (per well) were transferred into the 384 black well plate into wells B16, B17, B18.

For Control Five: Buffer Only

40 μl of buffer (per well) were transferred to the 384 black well plate into B19, B20, B21.

The plates were incubated at room temperature and covered in foil for 1 hour. The plates were then read on the Perkin Elmer plate reader using the FAM protocol (according to the manufacturer's instructions).

From the raw data, the buffer blank was subtracted for both the parallel and perpendicular values. The first values (S) are the parallel values. The average of the blank wells was subtracted from the triplicate average of each point in the curve. The process was repeated for the (P) perpendicular values. The average of the blank wells was subtracted from the triplicate averages of each point in the curve. Then, the mP was calculated as follows:

(S−P)/(P−S)×1000=mP

The results of this assay, shown in FIG. 3, demonstrate that the transcription factor modulators ARE and ARE-MPS are able to bind to their target DNA binding site, while the control compounds comprising the scrambled ARE sequence do not bind the DNA binding site.

### Example 3

Ability of ARE-MPS to Bind Full Length Androgen Receptor

To test the ability of the Transcription Factor Modulators of the present invention to bind to their target DNA binding site, an androgen responsive element having the sequence 5′-AGT CTG GTA CAG GGT GTT CTT TTT A-3′ (SEQ ID NO: 23) was attached to biotin. The ability of the transcription factor modulator of Examples 1 and 2 (ARE-MPS) and the ARE alone to antagonize the interaction of biotin-ARE with full-length native androgen receptor DNA binding site was compared.

Preparation of Nuclear Extract

The buffers used for these experiments are set forth below:

| | | | |
|---|---|---|---|
| Buffer A: | 10 mM Hepes KOH p7.9 | Buffer B: | 20 mM Hepes |
| | | | KOH pH7.9 |
| | 1.5 mM MgCl2 | | 1.5 mM MgCl2 |
| | 10 mM KCl | | 420 mM KCl |
| | 1 mM DTT | | 25% glycerol |
| | 0.2 mM PMSF | | 1 mM DTT |
| | | | 0.2 mM PMSF |

LnCAP cells were grown in T175 flasks in complete media until confluent. 1 μM dihydrotestosterone (DHT) was then added to each flask and the flasks were incubated for 1 hour. The cells were trypsinized off the flask and washed two times with cold phosphate-buffered saline (PBS). The cells were then resuspended in PBS and aliquoted into eppendorf tubes. The cells were then centrifuged in the cold on maximum speed for 10 seconds. The PBS was then removed and the pellet was resuspended in 0.5 mL Buffer A containing. The suspension was incubated on ice for 10 minutes. Each tube was vortexed for 30 seconds and then centrifuged in the cold at maximum speed for 30 seconds. The supernatant was removed by aspiration and the pellet was resuspended in 150 μL Buffer B. The suspension was incubated on ice for 20 minutes, and each tube was vortexed for 30 seconds, then centrifuged in the cold at maximum speed for 1 minute. The supernatant, which contained the nuclear extract, was collected, aliquoted into tubes at 100 μL/tube and frozen at −80° C. The protein concentration of the aliquots was then determined using Pierce Micro BCA Assay (Pierce Catalog #23235)

Pull Down of Androgen Receptor

Buffers used in the following procedure are set forth below:

Binding Buffer (BB): 15 mM HEPES, pH 7.9, 50 mM KCl, 2.5 mM MgCl₂, 5% glycerol, 0.1% triton-X, 1 mM DTT, 10 ug/mL poly dI/dC (Sigma Cat. #P-4929), 0.1 mg/mL AcBSA (Invitrogen, Cat. #15561-020), and protease inhibitors (Roche #1836145).

Wash Buffer: 15 mM HEPES, pH 7.9, 50 mM KCl, 2.5 mM MgCl₂, 5% glycerol, 0.1% triton-X, 1 mM DTT, 10 ug/mL poly dI/dC (Sigma Cat. #P-4929), and protease inhibitors (Roche #1836145).

Biotin-labeled ARE was prepared by annealing equimolar amounts of 5′-biotin-AGT CTG GTA CAG GGT GTT CTT TTT A-3′ and 5′-TAA AAA GAA CAC CCT GTA CCA GAC T-3′) in 50 mM NaCl, 100 mM Hepes, pH 7.2. ARE and MPS-ARE were prepared by annealing equimolar amounts of 5′-AGT CTG GTA CAG GGT GTT CTT TTT A-3′ or 5′-MPS-AGT CTG GTA CAG GGT GTT CTT TTT A-3′, and 5′-TAA AAA GAA CAC CCT GTA CCA GAC T-3′) in 50 mM NaCl, 100 mM Hepes, pH 7.2. The solutions were heated to 95° C. for 2 minutes and then allowed to cool at room temp for 1 hour. The biotin-ARE was diluted to a concentration of 0.5 pmoles/30 μL. 36 μg nuclear extract was then added to 500 μL 1× BB. Various concentrations of ARE or MPS-ARE were added as indicated. 0.5 pmole of biotin-ARE was added to each tube and the tubes were incubated for 1 hour in the cold on a rotator. 20 μL of Streptavidin Dynabeads M-280 (IGEN #402-175-02) was added to each tube and the tubes were incubated in the cold for 1 hour on rotator. The beads were washed 3 times with wash buffer using a magnet. The tubes were changed with each wash. The pellet from the last wash was resuspended in 25 μL of 1× NuPage LDS Sample Buffer (4× buffer Invitrogen Cat. #NP0007) containing 1× NuPage Sample Reducing Agent (10× Reducing Agent, Invitrogen Cat. #NP0004). Each sample was run on a 10% Bis Tris gel (10 well gel, Invitrogen, Cat. #NP0301) using 1× MOPS Buffer (20× MOPS, Invitrogen Cat. #NP0001). The gels were then subjected to Western blot and the blots were probed with anti-AR pAb (Santa Cruz, Cat. #sc-816).

Results

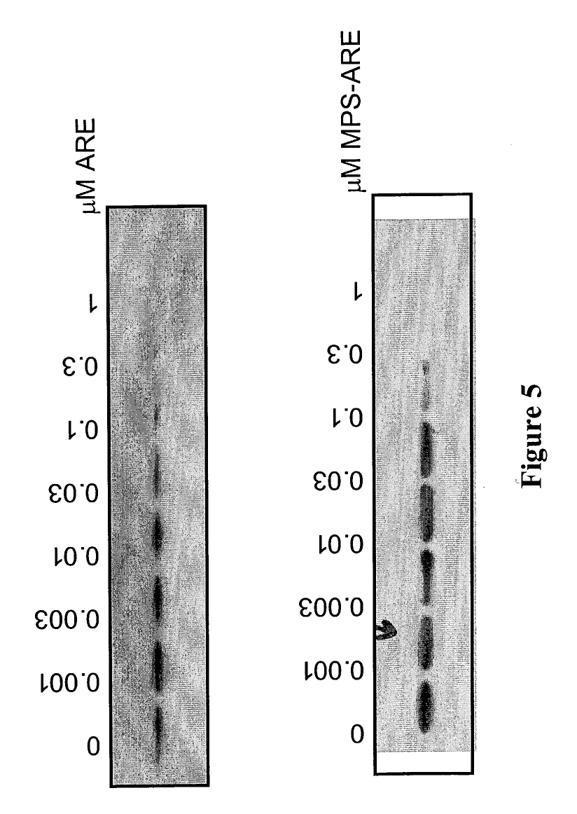

The results are shown in FIG. 5, which demonstrates that ARE and ARE-MPS compounds have a similar ability to compete with biotin-ARE for binding to native androgen receptor in the nuclear extract.

### Example 4

Down-Regulation of Androgen Receptor Responsive Gene Expression by ARE

LNCaP cells were removed from flasks with trypsin, washed in serum-free medium, resuspended to 2×10⁶ cells/ml and placed on ice. 0.5 ml cells were added to BTX 4 mm gap electroporation cuvettes (BTX, model 640). Samples were pulsed on a Biorad GenePulser at 0.25 kV, 960 μF, and 100 Ω. Cells were centrifuged, resuspended in complete medium containing 10 nM DHT and incubated for 4 hours at 37° C. Total RNA was isolated from the cells using Trizol® (Life Technologies, Inc., cat#15596-018) according to manufactures instructions. The RNA concentration was then estimated by use of RiboGreen® RNA Quantitation Kit, (Molecular Probes, Inc., cat # R-11490), according to manufactures instructions. cDNA synthesis was performed by use of Omniscript® RT kit (Qiagen Inc, cat# 205113), essentially according to manufacturer's instructions; to a reaction mixture (20 uL) comprising 1× Omniscript® RT Buffer, 0.2 units/μl Omniscript® Reverse Transcriptase (included in the kit), 1 μM Oligo-dT (18 mer) primer, and 0.5 units/μl RNase inhibitor (Roche Inc, cat# 799 0170) was added 0.2-1 μg RNA. The reaction mixture was allowed to incubate at 37° C. for one hour and stopped by heat inactivation of the enzyme for 5 minutes at 93° C. Real time PCR was performed on a Rotorgene2000 (Corbett Research, Australia) by use of QuantiTect SYBR Green PCR Kit (Qiagen Inc., cat# 204143) essentially according to manufacturer's instructions; using a reaction mixture (20 uL) comprising 1× QuantiTect SYBR Green PCR Master Mix, 0.01 units/μl uracil-DNA-glycosylase (Roche Inc., cat#1 775 375), 0.3 μM forward primer, 0.3 μM backward primer and 2 μL cDNA reaction mixture diluted 20 fold with water. The reaction was heated according to the following profile: 50° C. for 2 min., 95° C. for 15 min., 40 cycles of step 1; 95° C. for 15 sec., step 2; 55° C. for 30 sec and step 3; 72° C. for 30 sec. Fluorescence was acquired at the end of each step 3. The steady state level of transcripts (PSA II) from the androgen responsive gene, kallikrein 3, were measured with forward primer pri251 [CCCTGACTGTCAAGCTGAG] and backward primer pri252 [GGTGTGGGAGTGAGGACTG] and normalized to the steady state level of transcripts (actin) from the house keeping gene beta-actin, which were measured with forward primer pri157 [CAAGATCATTGCTCCTCCTG] and backward primer pri159 [CGTCATACTCCTGCTTGCTG]. Data was analyzed using the Rotorgene software.

Results

The results are set forth in FIG. 5 and show that steady state levels of PSA II were induced upon DHT treatment (compare no oligo, −DHT with 0 μM ARE, +DHT or 0 μM scram ARE, +DHT). ARE at 1, 5 or 20 μM was cble to reduce this induction, whereas this was not observed for the scrambled ARE at the same concentrations. This indicates that ARE is able to downregulate the expression of the androgen receptor responsive gene. Casodex was included in the study as a positive control as a known androgen receptor antagonist.

Equivalents

Those skilled in the art will recognize, or be able to ascertain using no more than routine experimentation, many equivalents to the specific embodiments and methods described herein. Such equivalents are intended to be encompassed by the scope of the following claims.

What is claimed is:

1. A transcription factor modulator comprising:
a transcription control recognition sequence for a transcription factor coupled to a membrane permeable peptidic sequence, wherein said membrane permeable peptidic sequence facilitates the delivery of said transcription control recognition sequence to a nucleus of a cell.

2. The transcription factor modulator of claim 1, wherein said transcription control recognition sequence comprises a promoter element.

3. The transcription factor modulator of claim 2, wherein said promoter element is selected from the group consisting of a CACCC-Box, a GC-Box, and a CAT-Box.

4. The transcription factor modulator of claim 1, wherein said transcription control recognition sequence comprises a hormone response element.

5. The transcription factor modulator of claim 4, wherein said hormone response element is selected from the group consisting of the androgen response element, the estrogen response element, the glucocorticoid response element, the thyroid hormone response element, the thyroid hormone inhibitory element, and the sterol-dependent repressor.

6. The transcription factor modulator of claim 5, wherein said androgen response element comprises a sequence selected from the group consisting of AGTACGTGATGTTCT (SEQ ID NO: 10), GAAACAGCCTGTTCT (SEQ ID NO: 11), AGCACTTGCTGTTCT (SEQ ID NO: 12), ATAGCATCTTGTTCT (SEQ ID NO: 13), AGTCCCACTTGTTCT (SEQ ID NO: 14), AGTACTTGTTGTTCT (SEQ ID NO: 15), AGCTCAGCTTGTACT (SEQ ID NO: 16), AGAACAACCTGTTGA (SEQ ID NO: 17), TGAAGTTCCTGTTCT (SEQ ID NO: 18), GTAAAGTACTCCAAGAA (SEQ ID NO: 19), GGAACAGCAAGTGCT (SEQ ID NO: 20) and 5′-AGT CTG GTA CAG GGT CTT TTT A-3′ (SEQ ID NO: 23).

7. The transcription factor modulator of claim 1, wherein said transcription control recognition sequence comprises an NF-κB binding site.

8. The transcription factor modulator of claim 7, wherein said NF-κB binding site comprises the sequence GGGRNNTYCC (SEQ ID NO: 21).

9. The transcription factor modulator of claim 1, wherein said transcription control recognition sequence comprises a double stranded oligodeoxynucleotide.

10. The transcription factor modulator of claim 1, wherein said transcription control recognition sequence comprises a peptide-nucleic acid.

11. The transcription factor modulator of claim 1, wherein said transcription control recognition sequence comprises a ribonucleic acid.

12. The transcription factor modulator of claim 1, wherein said transcription control recognition sequence comprises a modified phosphodiester bond.

13. The transcription factor modulator of claim 11, wherein said modified phosphodiester bond is selected from the group consisting of phosphorothioate, phosphoramidite, and methyl phosphate derivatives.

14. The transcription factor modulator of claim 1, wherein said transcription control recognition sequence consists of 5-30 nucleotides.

15. The transcription factor modulator of claim 1, wherein the membrane permeable peptidic sequence consists of 5-20 amino acid residues.

16. The transcription factor modulator of claim 1, wherein the membrane permeable peptidic sequence consists of 10-20 amino acid residues.

17. The transcription factor modulator of claim 1, wherein the membrane permeable peptidic sequence comprises at least five basic amino acid residues.

18. The transcription factor modulator of claim 1, wherein the membrane permeable peptidic sequence is selected from the group consisting of the Kaposi FGF signal sequence and sequences derived therefrom, the third helix of the antennapedia homeodomain and sequences derived therefrom, the HIV-1 Tat protein and sequences derived therefrom, and the gelsolin sequence and sequences derived therefrom.

19. The transcription factor modulator of claim 1, wherein the membrane permeable peptidic sequence comprises at least one D-amino acid residue.

20. The transcription factor modulator of claim 1, wherein said transcription control recognition sequence and said membrane permeable peptidic sequence are coupled via a linker.

21. The transcription factor modulator of claim 1, wherein said transcription control recognition sequence and said membrane permeable peptidic sequence are coupled directly.

22. A pharmaceutical composition comprising a therapeutically effective amount of a transcription factor modulator of claim 1 and a pharmaceutically acceptable carrier.

23. A method for modulating gene transcription in a cell comprising contacting said cell with a transcription factor modulator of claim 1 in a sufficient amount to modulate gene transcription in said cell.

24. A method of treating a subject suffering from a transcription factor-associated disorder, comprising administering to the subject a therapeutically effective amount of a transcription factor modulator of claim 1, thereby treating a subject suffering from a transcription factor-associated disorder.

25. The method of claim 24, wherein said transcription factor-associated disorder is cancer.

26. The method of claim 25, wherein said transcription factor-associated disorder is a hormonally responsive cancer.

27. The method of claim 26, wherein said transcription factor-associated disorder is prostate cancer.

28. The method of claim 26, wherein said transcription factor-associated disorder is breast cancer.

29. The method of claim 26, wherein said transcription factor-associated disorder is uterine cancer.

31. The method of claim 26, wherein said transcription factor-associated disorder is benign prostatic hypertrophy.

31. The method of claim 24, wherein said transcription factor-associated disorder is an NF-κB associated disorder.

32. The method of claim 31, wherein said NF-κB associated disorder is selected from the group consisting of inflammatory diseases, autoimmune diseases, ischemic diseases, cachexia, and cancer metastasis and invasion.

33. A transcription factor modulator comprising the sequence YARAARRAARRG coupled to the sequence 5′-AGT CTG GTA CAG GGT CTT TTT A-3′ (SEQ ID NO: 23).

34. A transcription control modulator comprising an androgen response element nucleotide sequence.

35. The transcription control modulator of claim 34, wherein the nucleotide sequence is conjugated to at least one modifying moiety.

36. The transcription control modulator of claim 34, wherein the androgen response element sequence is selected from the group consisting of AGTACGTGATGTTCT (SEQ ID NO: 10), GAAACAGCCTGTTCT (SEQ ID NO: 11), AGCACTTGCTGTTCT (SEQ ID NO: 12), ATAGCATCTTGTTCT (SEQ ID NO: 13), AGTCCCACTTGTTCT (SEQ ID NO: 14), AGTACTTGTTGTTCT (SEQ ID NO: 15), AGCTCAGCTTGTACT (SEQ ID NO: 16), AGAACAACCTGTTGA (SEQ ID NO: 17), TGAAGTTCCTGTTCT (SEQ ID NO: 18), GTAAAGTACTCCAAGAA (SEQ ID NO: 19), GGAACAGCAAGTGCT (SEQ ID NO: 20) and 5′-AGT CTG GTA CAG GGT CTT TTT A-3′ (SEQ ID NO: 23).


SEQUENCE LISTING (23 sequences)

SEQ ID NO: 1 - DNA, 9 bp, Artificial Sequence
  CACCC-Box
gccacaccc

SEQ ID NO: 2 - DNA, 10 bp, Artificial Sequence
  GC-Box
krggcgkrry

SEQ ID NO: 3 - DNA, 15 bp, Artificial Sequence
  CAT-Box
ttggcnnnnngccaa

SEQ ID NO: 4 - DNA, 15 bp, Artificial Sequence
  CAT-Box
ttggcnnnnnngcca

SEQ ID NO: 5 - DNA, 13 bp, Artificial Sequence
  Estrogen response element
ggtcacagtgacc

SEQ ID NO: 6 - DNA, 16 bp, Artificial Sequence
  Glucocorticoid response element
yggtwcamwntgtyct

SEQ ID NO: 7 - DNA, 18 bp, Artificial Sequence
  Thyroid hormone response element
aggtaagatcagggacgt

SEQ ID NO: 8 - DNA, 16 bp, Artificial Sequence
  Thyroid hormone inhibitory element
agggtataaaaagggc

SEQ ID NO: 9 - DNA, 8 bp, Artificial Sequence
  Sterol-dependent repressor
gtgsggtg

SEQ ID NO: 10 - DNA, 15 bp, Artificial Sequence
  Androgen response element
agtacgtgatgttct

SEQ ID NO: 11 - DNA, 15 bp, Artificial Sequence
  Androgen response element
gaaacagcctgttct

SEQ ID NO: 12 - DNA, 15 bp, Artificial Sequence
  Androgen response element
agcacttgctgttct

SEQ ID NO: 13 - DNA, 15 bp, Artificial Sequence
  Androgen response element
atagcatcttgttct

SEQ ID NO: 14 - DNA, 15 bp, Artificial Sequence
  Androgen response element
agtcccacttgttct

SEQ ID NO: 15 - DNA, 15 bp, Artificial Sequence
  Androgen response element
agtacttgttgttct

SEQ ID NO: 16 - DNA, 15 bp, Artificial Sequence
  Androgen response element
agctcagcttgtact

SEQ ID NO: 17 - DNA, 15 bp, Artificial Sequence
  Androgen response element
agaacaacctgttga

SEQ ID NO: 18 - DNA, 15 bp, Artificial Sequence
  Androgen response element
tgaagttcctgttct

SEQ ID NO: 19 - DNA, 17 bp, Artificial Sequence
  Androgen response element
gtaaagtactccaagaa

SEQ ID NO: 20 - DNA, 15 bp, Artificial Sequence
  Androgen response element
ggaacagcaagtgct

SEQ ID NO: 21 - DNA, 10 bp, Artificial Sequence
  NF-kappa B
gggrnntycc

SEQ ID NO: 22 - DNA, 17 bp, Artificial Sequence
  Androgen response element
ggaatacannntgttct

SEQ ID NO: 23 - DNA, 21 bp, Artificial Sequence
  Androgen response element
agtctggtacagggtcttttt